(19) United States
(12) Patent Application Publication
(10) Pub. No.: US 2023/0031808 A1
(43) Pub. Date: Feb. 2, 2023

(54) LATERAL FLOW IMMUNOASSAY DEVICE WITH INCREASED DETECTION SIGNAL INTENSITY AND METHOD FOR DETECTING TARGET MATERIAL USING THE SAME

(71) Applicant: Pukyong National University Industry-University Cooperation Foundation, Busan (KR)

(72) Inventors: Joong Ho Shin, Basan (KR); Se Been Park, Busan (KR)

(21) Appl. No.: 17/855,035

(22) Filed: Jun. 30, 2022

(30) Foreign Application Priority Data
Jul. 1, 2021 (KR) 10-2021-0086452

(51) Int. Cl. B01L 3/00; G01N 33/53; G01N 33/558

(52) U.S. Cl. CPC B01L 3/5023; G01N 33/5308; G01N 33/558; B01L 2300/0825

(57) ABSTRACT

The present disclosure relates to a lateral flow immunoassay device with an increased detection signal intensity by applying a pressure to a membrane, and the lateral flow immunoassay device manufactured by applying the pressure to the membrane according to the present disclosure shows significantly higher sensitivity than that of a lateral flow immunoassay device without applying a pressure to the membrane. Accordingly, the present disclosure, as an invention that increases the detection signal intensity by a very simple physical method without the need for chemical treatment, is suitable for mass production and may obtain an effect of reducing costs without requiring the use of additional materials. Therefore, the present disclosure can be usefully used for a lateral flow immunoassay device for detecting various target materials.

### CROSS-REFERENCE TO RELATED APPLICATIONS

This application is based on and claims priority from Korean Patent Application No. 10-2021-0086452, filed on Jul. 1, 2021, with the Korean Intellectual Property Office, the disclosure of which is incorporated herein in its entirety by reference.

### TECHNICAL FIELD

The present disclosure relates to a lateral flow immunoassay device with an increased detection signal intensity, and more particularly, to a lateral flow immunoassay device with an increased detection signal intensity by reducing the flow rate of a sample using a pressure, and a method for detecting a target material using the same.

### BACKGROUND

Diagnostic methods and diagnostic tools made by qualitative or quantitative analysis of trace substances contained in a biopsy such as blood or urine have been developed. Since radioimmunoassay (MA) using radioactive isotopes was first introduced in the 1950s, enzyme-linked immunosorbent assay (ELISA) was developed and advanced in the 1970s and 1980s. Currently, the ELISA is one of the most used methods and has become an essential tool in researches in medicine or life sciences.

However, typical immunoassays including MA and ELISA usually quantify a type of analyte per sample through a complex multi-step process using expensive analytical equipment. Therefore, it is not easy to use the immunoassays in small hospitals, emergency rooms, homes, etc. that are not equipped with such facilities or equipment. A diagnostic product designed to compensate for this weakness is a simple diagnostic kit using immunochromatography.

A representative type of immunochromatographic assay may include lateral flow assay (LFA). A kit structure of the LFA type consists of a sample pad to which a sample is applied, a conjugate pad or releasing pad on which a detection antibody is coated, a developing membrane (mainly, nitrocellulose) or strip on which the sample moves and is separated and an antibody-antigen reaction occurs, and an absorption pad for continuously moving the sample.

The detection antibody is bound to, for example, colloidal gold particles for indication. Instead of the gold particles, latex beads or carbon particles are also used. A diagnostic kit for lateral flow assay is designed to detect an analyte, usually in sandwich form. That is, while the analyte in a liquid sample is applied to the sample pad and begins to move, first, the analyte reacts with the detection antibody that is non-fixedly coated on the releasing pad (or conjugate pad) to be continuously developed in the form of an antigen-antibody conjugate. While moving, the analyte reacts once more with a capture antibody fixed on the developing membrane to form a sandwich-type complex. Since the capture antibody is immobilized on the developing membrane, if the antigen-antibody reaction continuously occurs, the complex is accumulated on an immobilization surface of the capture antibody. Since proteins are transparent to the naked eye, whether the complex is formed and the relative amount are determined by the color development intensity of the attached gold particles. Such LFA has been widely used in various fields such as pregnancy diagnosis, cancer diagnosis, and microorganism detection, and may make diagnoses very simple.

However, since the determination is made with the naked eye, there is a problem in that accurate diagnosis is difficult if the color development intensity is weak for various reasons, such as a small amount of a target material included in a biological sample.

Therefore, in order to solve this problem, a wax pillar has been developed to pattern solid ink on a membrane surface using wax ink printing technology and increasing the detection signal intensity by melting the solid ink to hinder the fluid flow on the membrane. The technique showed a result of increasing the binding time between the immunocomplex and the detection antibody and increasing the signal intensity of the LFA through various types of wax pillar patterns. However, there are cases where the wax patterning is not uniform, and there is a disadvantage that heating needs to be performed after patterning. In addition, there was an attempt to increase the signal intensity by introducing silver staining to the LFA kit test. However, this method has a disadvantage in that an additional step for staining is required, and a design of the device becomes more complicated to automate the staining.

Therefore, it is necessary to develop a lateral flow immunoassay device that enhances the detection signal intensity by a simpler method.

### SUMMARY

The present inventors have made studies to increase the detection signal intensity in a lateral flow immunoassay device and then found that the detection signal intensity was increased by reducing a pore size of a membrane using a pressure, and then completed the present disclosure.

Therefore, the present disclosure has been made in an effort to provide a lateral flow immunoassay device with an increased detection signal intensity.

The present disclosure has been also made in an effort to provide a method for detecting a target material.

An exemplary embodiment of the present disclosure provides a lateral flow immunoassay device with an increased detection signal intensity including a sample pad for introducing a sample containing a target material; a conjugate pad including a first conjugate bound with a marker; a membrane of which a test line to which a second conjugate is immobilized and a control line to which a third conjugate is immobilized are located on the surface at an interval; and an absorption pad.

Another exemplary embodiment of the present disclosure provides a method for detecting a target material, including introducing a sample containing a target material into a sample pad of a lateral flow immunoassay device according to the present disclosure.

According to the exemplary embodiment of the present disclosure, the present disclosure relates to a lateral flow immunoassay device with an increased detection signal intensity by applying a pressure to a membrane, and the lateral flow immunoassay device manufactured by applying the pressure to the membrane according to the present disclosure shows significantly higher sensitivity than that of a lateral flow immunoassay device without applying a pressure to the membrane. Accordingly, the present disclosure, as an invention that increases the detection signal intensity by a very simple physical method without the need for chemical treatment, is suitable for mass production and may obtain an effect of reducing costs without requiring the use of additional materials. Therefore, the present disclosure can be usefully used for a lateral flow immunoassay device for detecting various target materials.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 is a schematic diagram illustrating a lateral flow immunoassay device according to the present disclosure.

FIG. 2A is a diagram for theoretically describing an effect of reducing the flow rate and increasing the sensitivity according to a pressure to the membrane in the lateral flow immunoassay device according to the present disclosure.

FIG. 2B is also a diagram for theoretically describing an effect of reducing the flow rate and increasing the sensitivity according to a pressure to the membrane in the lateral flow immunoassay device according to the present disclosure.

FIG. 3 is a result of confirming the flow rate of a sample according to a pressure by applying a pressure of 20, 40, 60, or 80 kgf to a partial region of the membrane in the lateral flow immunoassay device according to the present disclosure.

FIG. 4 is a result of confirming the flow rate of a sample according to a pressure by applying a pressure of 120 or 160 kgf to a partial region of the membrane in the lateral flow immunoassay device according to the present disclosure.

FIG. 5 is a result of confirming the sensitivity according to a pressure in a lateral flow immunoassay device for detecting C-reactive protein (CRP) according to the present disclosure.

FIGS. 6 and 7 are results of confirming the sensitivity according to a pressure in a lateral flow immunoassay device for detecting SARS-CoV-2 spike protein peptide according to the present disclosure.

FIGS. 8A and 8B illustrate images showing positions where the pressure is applied (a gray box illustrates a pressed position) in a lateral flow immunoassay device. FIG. 8A illustrates a result of a lateral flow immunoassay device in which a pressure is applied on a test line. FIG. 8B illustrates a result of a lateral flow immunoassay device in which the pressure is applied between the test line and a control line. FIG. 8C illustrates results of comparing detection signals according to a position where the pressure is applied.

### DETAILED DESCRIPTION

In the following detailed description, reference is made to the accompanying drawing, which forms a part hereof. The illustrative embodiments described in the detailed description, drawing, and claims are not meant to be limiting. Other embodiments may be utilized, and other changes may be made, without departing from the spirit or scope of the subject matter presented here.

Hereinafter, the present disclosure will be described in detail.

The present disclosure provides a lateral flow immunoassay device with an increased detection signal intensity including a sample pad for introducing a sample containing a target material; a conjugate pad including a first conjugate bound with a marker; a membrane of which a test line to which a second conjugate is immobilized and a control line to which a third conjugate is immobilized are located on the surface at an interval; and an absorption pad.

The present disclosure is characterized in that a pore size is reduced by applying a pressure to the membrane between the test line and the control line. That is, it is characterized that the flow rate is reduced by increasing the resistance in the flow of the sample by reducing the pore size.

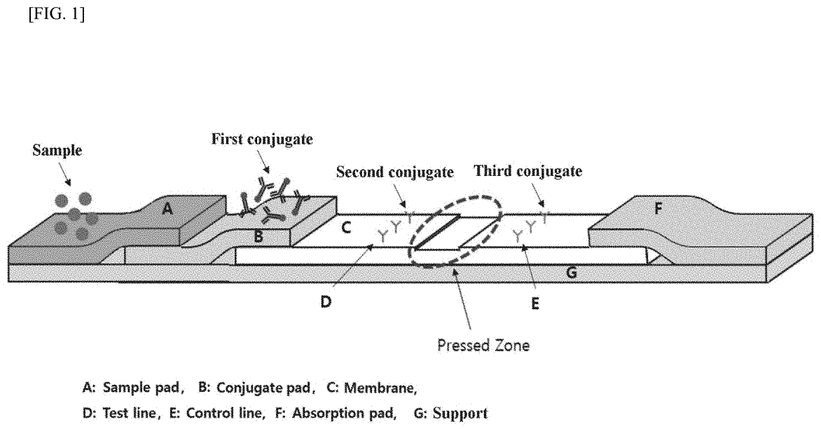

The lateral flow immunoassay device of the present disclosure may have a structure in which the sample pad, the conjugate pad, the membrane, and the absorption pad are sequentially connected onto a support as illustrated in FIG. 1.

In the present disclosure, the “membrane” may be formed of any variety of materials through which the sample may pass. For example, the membrane may be formed of materials of natural, synthetic, or synthetically modified naturally occurring materials, such as polysaccharides (e.g., cellulose material, paper, and cellulose derivatives such as cellulose acetate and nitrocellulose); polyether sulfone; polyethylene; nylon; polyvinylidene fluoride (PVDF); polyester; polypropylene; silica; inorganic materials uniformly dispersed in a porous polymer matrix with a polymer such as vinyl chloride, vinyl chloride-propylene copolymer, and vinyl chloride-vinyl acetate copolymer, such as inactivated alumina, diatomaceous earth, MgSO4, or other fine inorganic materials; naturally occurring (e.g., cotton) and synthetic (e.g., nylon or rayon) fabrics; porous gels, such as silica gel, agarose, dextran, and gelatin; polymeric films such as polyacrylamide, and the like. Preferably, the materials include polymeric materials, such as nitrocellulose, polyethersulfone, polyethylene, nylon, polyvinylidene fluoride, polyester, and polypropylene, but are not limited thereto.

In the present disclosure, the “sample pad” refers to a pad capable of diffusion flow by accommodating a sample to be analyzed, and consists of a material having sufficient porosity to accommodate and contain the sample to be analyzed. Such a porous material includes fibrous paper, microporous membranes made of cellulose materials, cellulose, cellulose derivatives such as cellulose acetate, nitrocellulose, glass fibers, naturally occurring cotton, fabrics such as nylon, porous gels, or the like, but is not limited thereto. Further, the pore of the sample pad may serve to filter residual components (e.g., red blood cells) in the sample.

In the present disclosure, the “conjugate pad” includes a first conjugate bound with the marker, and the first conjugate may specifically bind only to the target material to form a complex. The conjugate pad may serve to keep the first conjugate functionally stable until a test is performed. Specifically, the first conjugate exists in a dried state and then the sample (or sample solution) containing the target material is absorbed to impart fluidity and moves to a detection area of the membrane material located downstream. To this end, for example, a conjugate buffer composition containing carbohydrates (e.g., sucrose) and a resolubilization agent may be used. In this case, when the conjugate particles are dried in the presence of sugars, the sugar molecules form a layer around the particles to be stabilized. As the sample is introduced into the conjugate pad, the sugar molecules are quickly dissolved to carry the first conjugate particles into the fluid flow.

In the present disclosure, the “absorption pad” may be located adjacent to or near the end of the membrane. The absorption pad generally receives a fluid sample moving through the entire membrane, and may help in promoting capillary action and diffuse flow of the fluid through the membrane. The absorption pad also absorbs and removes excess sample or buffer to prevent the disturbing of the results.

In the present disclosure, the “support” may be formed of any material as long as it may support and transport the sample pad, the membrane, and the absorption pad, but in general, it is preferable to be liquid impermeable so that the fluid of the sample that diffuses through the membrane does not leak through the support. For example, the material may include glass; and polymeric materials such as polystyrene, polypropylene, polyester, polybutadiene, polyvinylchloride, polyamide, polycarbonate, epoxide, methacrylate, and polymelamine, but is not limited thereto.

According to an exemplary embodiment of the present disclosure, as a result of applying a pressure of 0, 20, 40, 60, or 80 kgf to a partial region of the membrane and confirming the flow rate, it was confirmed that the flow rate was decreased as the pressure was increased.

In addition, as a result of confirming a change in the flow rate after applying a pressure of 120 or 160 kgf to the partial region of the membrane, similarly, it was confirmed that the flow rate was decreased in proportion to the increased pressure, but the reproducibility deteriorated.

Accordingly, the present disclosure is characterized by applying a pressure of 20 to 110 kgf to the membrane between the test line and the control line. More preferably, a pressure of 20 to 80 kgf may be applied, and even more preferably, a pressure of 40 kgf may be applied.

In addition, the interval does not affect the test result, that is, the detection signal intensity, but is preferably a distance enough to visually confirm the results of the test line and the control line at once. In an exemplary embodiment of the present disclosure, the pressure was applied between the test line and the control line at an interval of 6 mm.

The interval may be 2 to 20 mm, preferably 2 to 10 mm, more preferably 3 to 7 mm, even more preferably 6 mm, but is not limited thereto.

When the pressure is applied to the entire membrane, all pores in the membrane are deformed, and then the flow rate of the sample may be reduced beyond what is desired. Therefore, the present disclosure improves the sensitivity by controlling the pressure applied to the membrane between the test line and the control line to increase the binding probability of the target material of the complex to which the first conjugate is bound and the second conjugate immobilized to the test line.

In the present disclosure, the first conjugate, the second conjugate, or the third conjugate may be at least one selected from the group consisting of an antibody, an antigen, a nucleic acid aptamer, a hapten, an antigen protein, a DNA, a DNA-binding protein, and a hormone-receptor, but is not limited thereto.

In addition, the marker may be at least one selected from the group consisting of nanoparticles, chromogenic enzymes, and fluorescent materials, but is not limited thereto.

In the present disclosure, the “nanoparticle” refers to a nanoparticle that serves as a detectable marker. The nanoparticles may be any one selected from the group consisting of noble metals of gold (Au), silver (Ag), platinum (Pt), palladium (Pd), iridium (Ir), rhodium (Rh), and ruthenium (Ru); Titanium (Ti), zirconium (Zr), hafnium (Hf), vanadium (V), niobium (Nb), tantalum (Ta), chromium (Cr), molybdenum (Mo), tungsten (W), ruthenium (Ru), osmium (Os); iron (Fe), nickel (Ni), cobalt (Co); magnesium oxide (MgO), titanium dioxide (TiO₂), vanadium pentoxide (V₂O₅), zinc oxide (ZnO), latex beads, magnetic beads, quantum dots, upconversion nanoparticles (UCNP), a graphene-nanoparticle complex, and color dyed particles, preferably gold (Au) nanoparticles or latex beads, most preferably gold (Au) nanoparticles, but are not limited thereto.

The binding between the conjugate and the marker may be a covalent bond or a non-covalent bond, and may include nucleic acid hybridization. Such a marker generates a detectable signal directly or indirectly related to the amount of the target material in the test sample.

In the present disclosure, the first conjugate and the second conjugate may specifically bind to the target material. Accordingly, the second conjugate may bind to the target material formed in the conjugate pad and the target material of the first conjugate complex according to the flow of the fluid. The amount of the complex binding to the second conjugate in the test line increases according to the amount of the target material, and the color development of the test line increases by a signal of the accumulated marker.

In addition, the third conjugate of the control line does not specifically bind to the target material, but may bind to the first conjugate. Accordingly, even if the first conjugate passes the test line because it is not bound to the target material, the first conjugate may bind to the third conjugate to be captured on the control line. Thus, the response in the control line indicates that the liquid sample is properly passing through a sensor (i.e., since the signal on the control line indicates that the first conjugate is present and the third conjugate binds to this first conjugate, an immune detection sensor is operating properly). As a result, when the target material is present, both the test line and the control line are colored, and two distinct lines may be identified. When there is no target material, only one control line is colored, and then qualitative analysis is possible.

In the present disclosure, the “target material” may be at least one selected from the group consisting of an antibody, an antigen, a nucleic acid aptamer, a hapten, an antigen protein, a DNA, a DNA-binding protein, a hormone, a tumor-specific marker, and a tissue-specific marker, but is not limited thereto.

In the present disclosure, the “sample” is used in the broadest sense. On the one hand, it is meant to include samples or cultures (e.g., microorganism cultures). On the other hand, it is meant to include both biological and environmental samples. In addition, the sample may include synthetic-origin samples. The biological sample may be at least one selected from the group consisting of whole blood, plasma, serum, sputum, tears, mucus, nasal washes, nasal aspirate, breath, urine, semen, saliva, peritoneal washings, ascites, cystic fluid, meningeal fluid, amniotic fluid, leukocytes, peripheral blood mononuclear cells, buffy coat, glandular fluid, pancreatic fluid, lymph fluid, pleural fluid, nipple aspirate, bronchial aspirate, synovial fluid, joint aspirate, organ secretions, cells, cell extract, and cerebrospinal fluid, but is not limited thereto.

In addition, the present disclosure provides a method for detecting a target material, including: introducing a sample containing a target material into a sample pad of a lateral flow immunoassay device according to the present disclosure.

Since the lateral flow immunoassay device according to the present disclosure exhibits an improved detection limit and an increased detection signal intensity, it is possible to accurately detect the target material even with a small amount of sample.

The above-described contents of the present disclosure are equally applied to each other unless otherwise contradict each other, and those appropriately modified and implemented by those skilled in the art are also included in the scope of the present disclosure.

Hereinafter, the present disclosure will be described in detail through Examples, but the scope of the present disclosure is not limited only to the Examples below.

### Example 1. Confirmation of Effect of Pressure Applied to Membrane

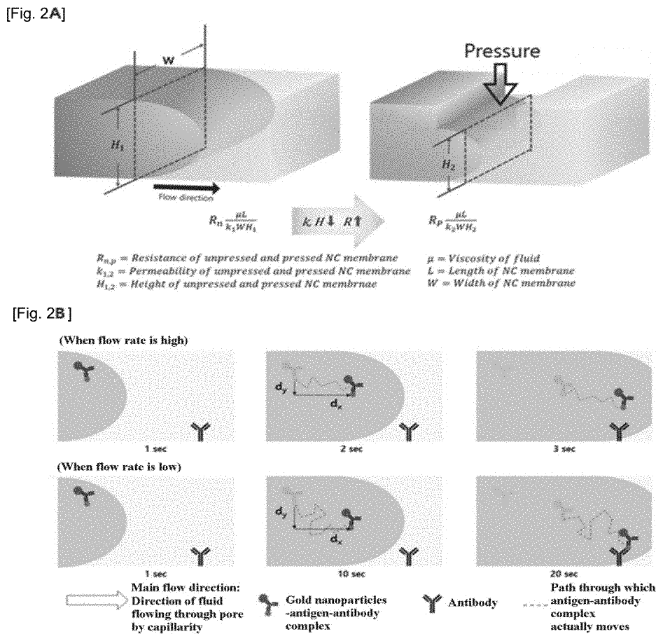

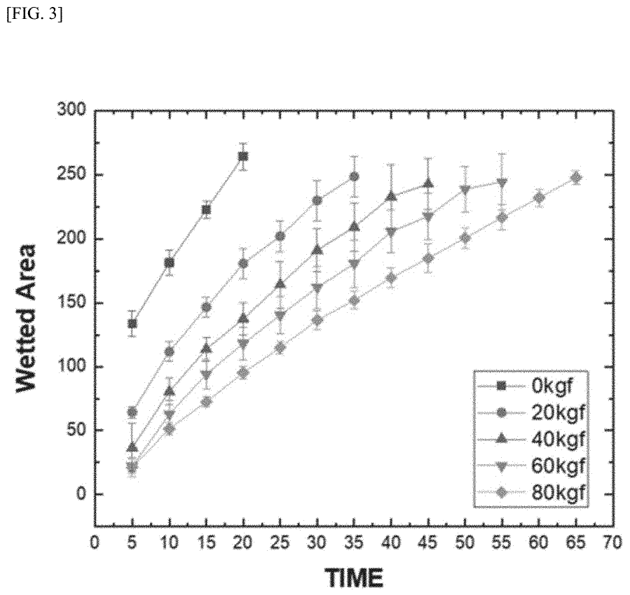

A pressure of 0, 20, 40, 60, or 80 kgf was applied to a partial region of a nitrocellulose membrane (mdi) (a region corresponding to 10 mm from the end of the membrane), and a solution in which a dye was dissolved in water was flowed to confirm the flow rate thereof. At this time, the flow rate was confirmed against the resistance calculated through Equation disclosed in FIG. 2A. More specifically, the resistance was calculated through Equation 1 below.

$${R}_{p} = \frac{μ L}{{K}_{2} {WH}_{2}} \qquad [ Equation 1 ]$$

In Equation 1, Rₚ represents the resistance of the pressed membrane, μ represents the viscosity of the sample solution, L represents the length of the membrane, K₂ represents the permeability of the pressed membrane, H₂ represents the height of the pressed membrane, and W represents the width of the membrane.

The pore size (diameter) of the membrane plays a major role in determining the flow rate of the sample. The larger the pore size, the higher the permeability and the higher the flow rate of the sample. Like the present disclosure, when the partial region of the membrane is pressed, as illustrated in FIG. 2A, the pores of the membrane are deformed inelastically, the pore size of an extruded part becomes small, and the fluid permeability also decreases. The reduced height H₂ and permeability K₂ of the membrane exerts resistance to the flowing liquid, thereby reducing the flow rate of the sample. This resistance value is proportional to the intensity of the initially applied pressure, and the resistance value and the flow rate may be adjusted simply and reproducibly by adjusting the pressure.

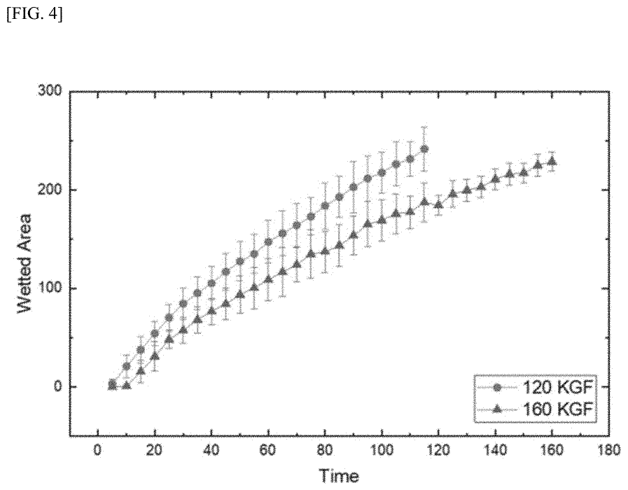

Therefore, as a result of applying a pressure of 20, 40, 60, or 80 kgf to the partial region of the membrane and confirming the flow rate of the sample according to the pressure, it was confirmed that the flow rate decreased as the pressure increased as illustrated in FIG. 3.

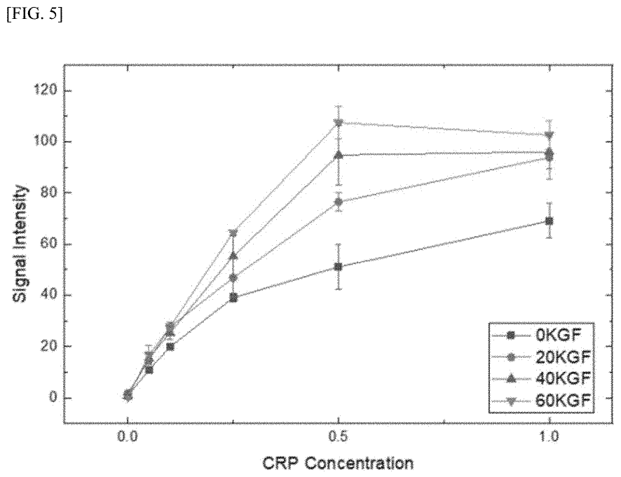

In addition, after a pressure of 120 or 160 kgf was greatly applied sequentially to the same region of the membrane, the decreased flow rate was confirmed and illustrated in FIG. 4. As a result, as illustrated in FIG. 3, it was confirmed that the flow rate decreased as the pressure was applied, and the decreased degree was larger than that of FIG. 3. However, when the pressure of 120 kgf or 160 kgf was applied, it was confirmed that the time required for the sample to flow from the sample pad to the absorption pad was further increased than the result of FIG. 3. Therefore, when the pressure of 120 kgf or more was applied, it was determined that a problem may be caused in that the time required for the sample to flow increased and the amount of evaporation of the sample increased during that time.

### Example 2. Manufacture of Lateral Flow Immunoassay Device for Each Target Material

2-1. Manufacture of Device for Detecting C-Reactive Protein (CRP)

2-1-1. Synthesis of Conjugates

A 0.5 M potassium carbonate buffer (K₂CO₃ buffer, pH 12) was added to 1.5 mL of a gold nanoparticle colloidal solution (Sigma Aldrich, 20 nm), and reacted with 2 mg/ml of an anti-CRP antibody (Abcam). Thereafter, 3% BSA diluted in phosphate buffer saline (PBS) was reacted to coat the surface of a first conjugate. The first conjugate having the surface coated with the BSA was centrifuged to remove a liquid in an upper layer except for a precipitate part. A suspension buffer [PBS, 3% BSA, 0.25% Tween 20] was added thereto to make the precipitate float in the solution.

2-1-2. Manufacture of Lateral Flow Immunoassay Device

GFDX (Millipore) was cut into 10×10 mm, and 60 μL of the conjugate prepared in Example 2-1-1 was applied to the surface of the GFDX and dried to prepare a conjugate pad.

In addition, a dispenser was used to form a test line and a control line on the nitrocellulose membrane (mdi). 1 mg/mL of an anti-CRP antibody (Abcam) was immobilized onto the test line, and 1 mg/mL of a goat anti-mouse IgG antibody (Sigma Aldrich) was immobilized onto the control line and then dried. At this time, the test line and the control line were fixed at an interval of 6 mm therebetween.

The membrane was placed on a jig manufactured by a 3D printer, and then placed on a load cell. A stamp and a jig manufactured by the 3D printer and acryl were assembled on a press machine, and the position of the load cell on which the jig was placed was adjusted so as to be pressed between the test line and the control line, and then the membrane was pressed.

The membrane was first attached to plastic coated paper, and the conjugate pad and an absorption pad (Ahlstrom-MunksjÖ) were laminated.

2-2. Manufacture of Device for Detecting SARS-CoV-2 Spike Protein Peptide

2-2-1. Synthesis of Conjugates

The conjugates were synthesized in the same manner as in Example 2-1-1 using 1 mg/ml of an anti-SARS-CoV-2 spike protein peptide antibody (Abcam) instead of 2 mg/ml of the anti-CRP antibody (Abcam).

2-2-2. Manufacture of Lateral Flow Immunoassay Device

The lateral flow immunoassay device was manufactured in the same manner as in Example 2-1-2 using an anti-SARS-CoV-2 spike protein peptide antibody (Abcam) instead of the anti-CRP antibody (Abcam) of the test line.

### Example 3. Confirmation of Effect According to Pressure of Lateral Flow Immunoassay Device According to Present Disclosure

3-1. Lateral Flow Immunoassay Device for Detecting CRP

When the sample is introduced into the LFA, the sample flows through the membrane via the conjugate pad, and a target in the sample forms a complex binding to the first conjugate during flowing. The flow is a convective flow, and the complex moves from one end of the membrane through the flow to the other end through the test line and the control line. While the convective flow occurs, diffusive transport of the complex due to diffusion also occurs in the pore of the membrane, but the movement randomly occurs by Brownian motion due to collision with water molecules, and a diffusion distance moving from the original position is increased over time due to the diffusion. A dₓ component in FIG. 2B is mainly determined by the convective transport (speed) of the main flow, and a d_{y} component is determined by the diffusive transport, but when the convective transport of the main flow occurs too quickly (if the flow rate is high), while the complex is transported in a dₓ direction, the time to be dispersed in a d_{y} direction is insufficient so that the probability of binding to the second conjugate immobilized onto the test line is reduced. On the contrary, if the convective transport of the main flow occurs slowly (when the flow rate is low), the time required to move by the same d_{y} distance increases, so that the time for the complex to move due to diffusive transport increases, and as a result, a diffusion distance of moving from the original position in the d_{y} direction is increased. Consequently, the probability of binding to the second conjugate immobilized onto the test line is increased.

The present disclosure has an effect of increasing an effective concentration by reducing the flow rate of the sample introduced to the LFA through the principle, thereby increasing the detection signal intensity and lowering the detection limit of the LFA.

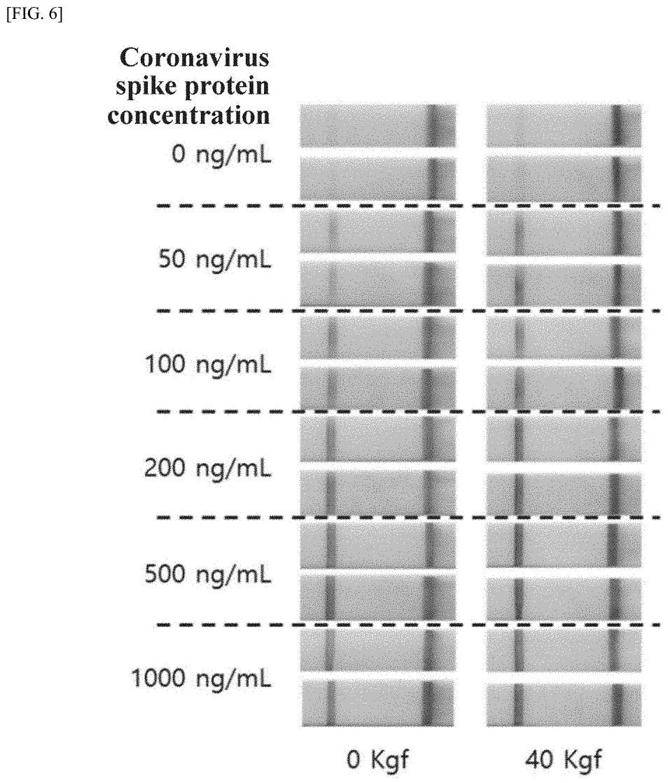

In order to actually confirm this effect, in the lateral flow immunoassay device for detecting the CRP in Example 2-1, the sensitivity according to the pressure applied to the membrane was confirmed. At this time, a sample containing each concentration (0, 0.5, or 10 μg/mL) of CRP in the sample was introduced into the sample pad of the device manufactured by applying a pressure of 20, 40, or 60 kgf to confirm the intensity of a color development signal of the test line. As a result, as illustrated in FIG. 5, it was confirmed that as the pressure applied to the membrane was increased, the intensity of the color development signal of the test line became stronger.

In addition, the detection limit was calculated by setting the CRP concentration of 0 to 0.25 μg/mL as a linear section. The detection limit was 0.00484 μg/mL in an experimental group of the unpressed membrane (0 kgf), but in an experimental group of a pressed membrane, it was confirmed that the detection limit was decreased to 0.0240, 0.0210, and 0.0298 μg/mL as the pressure was increased to 20, 40, or 60 kgf. This does not mean that the greater a force applied to the membrane, the lower the detection limit. Among compared conditions, it was confirmed that an experimental group to which a pressure of 40 kgf was applied showed about two times or more improved detection limit than an experimental group to which no pressure was applied.

3-2. Device for Detecting SARS-CoV-2 Spike Protein Peptide

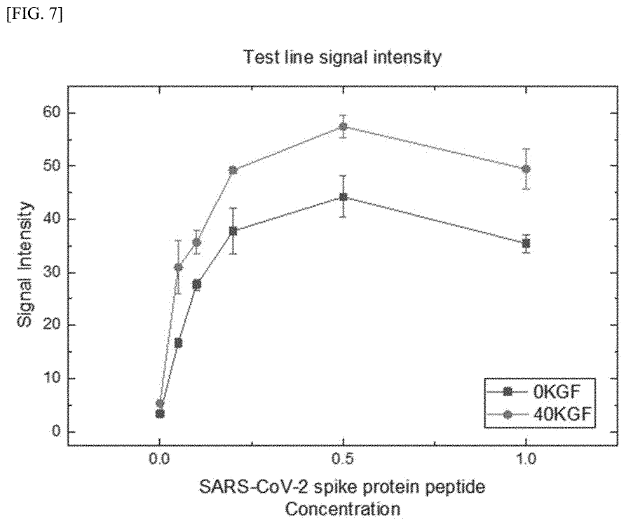

In addition, in the device for detecting the SARS-CoV-2 spike protein peptide in Example 2-2, the sensitivity according to the pressure applied to the membrane was confirmed. At this time, a sample containing each concentration (0, 50, 100, 200, 500, 1000 ng/mL) of the SARS-CoV-2 spike protein peptide in the sample was introduced into the sample pad of the device manufactured by applying a pressure of 0 or 40 kgf to the membrane to confirm the intensity of the color development signal of the test line. As a result, as illustrated in FIG. 6, it was confirmed that in the experimental group applying the pressure of 40 kgf to the membrane, the color development intensity became stronger. In addition, the detection signal of each experimental group was confirmed by administering a sample containing SARS-CoV-2 spike protein peptide at 0, 0.1, or 1 μg/mL, and as a result, as illustrated in FIG. 6, it was confirmed that the experimental group to which the pressure of 40 kgf was applied showed a stronger signal.

In addition, the detection limit was calculated by setting the SARS-CoV-2 spike protein peptide concentration of 0.05 to 0.2 μg/mL as a linear section. It was confirmed that in the case of the experimental group of the unpressed membrane (0 kgf), the detection limit was 0.0240 μg/mL, but the detection limit was decreased to 0.0178 μg/mL as the pressure of 40 kgf was applied to the membrane. As a result, it was confirmed that the experimental group to which the pressure of 40 kgf was applied to the membrane showed about 1.3 times or more improved detection limit than that of the experimental group to which no pressure was applied.

### Example 4. Confirmation of Effect According to Pressed Position of Lateral Flow Immunoassay Device According to Present Disclosure

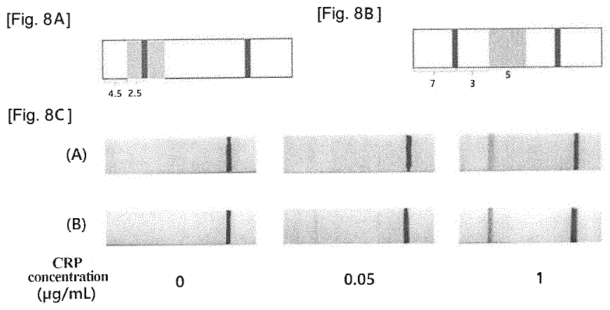

The sensitivity according to a position of the membrane to which the pressure was applied was confirmed in a lateral flow immunoassay device according to the present disclosure. To this end, the lateral flow immunoassay device for detecting the CRP in Example 2-1 was manufactured by applying the pressure of 40 kgf on the test line of the membrane or between the test line and the control line like the present disclosure (see FIG. 8A and FIG. 8B), and the detection intensity of each experimental group was compared.

As a result, as illustrated in FIG. 8C, it was confirmed that a lateral flow immunoassay device B manufactured by applying a pressure of 40 kgf between the test line and the control line of the present disclosure showed a stronger intensity of the detection signal than a lateral flow immunoassay device A manufactured by applying the same pressure on the test line. As a result, in the lateral flow immunoassay device of the present disclosure, it was confirmed that the position for applying the pressure to the membrane was most preferably between the test line and the control line.

Overall, the present disclosure relates to a lateral flow immunoassay device with the increased detection signal intensity by controlling the flow rate of the introduced sample through a change in pore size of the membrane. More specifically, it was confirmed that the change in the pore size of the membrane was caused by applying a pressure of 20 to 110 kgf to a region between the test line and the control line of the membrane and the sensitivity of the lateral flow immunoassay device manufactured by applying the pressure to the membrane was significantly increased compared to the unpressed device. Accordingly, the lateral flow immunoassay device with the increased detection signal intensity of the present disclosure, as an invention of increasing the detection signal intensity in a very simple physical method without a need for chemical treatment, is suitable for application in mass production, and may obtain an effect of reducing costs without requiring the use of additional materials. Therefore, the present disclosure can be usefully used in a lateral flow immunoassay device for detecting various target materials.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

What is claimed is:

1. A lateral flow immunoassay device with an increased detection signal intensity comprising:
a sample pad for introducing a sample containing a target material;
a conjugate pad including a first conjugate bound with a marker;
a membrane of which a test line to which a second conjugate is immobilized and a control line to which a third conjugate is immobilized are located on a surface at an interval; and
an absorption pad.

2. The lateral flow immunoassay device of claim 1, wherein a pore size is reduced by applying a pressure to the membrane therebetween.

3. The lateral flow immunoassay device of claim 2, wherein the pressure is 20 to 110 kgf.

4. The lateral flow immunoassay device of claim 1, wherein the first conjugate, the second conjugate, or the third conjugate is at least one selected from the group consisting of an antibody, an antigen, a nucleic acid aptamer, a hapten, an antigen protein, a DNA, a DNA-binding protein, and a hormone-receptor.

5. The lateral flow immunoassay device of claim 1, wherein the first conjugate and the second conjugate specifically bind to the target material, and the third conjugate binds to the first conjugate.

6. The lateral flow immunoassay device of claim 1, wherein the marker is at least one selected from the group consisting of nanoparticles, chromogenic enzymes, and fluorescent materials.

7. The lateral flow immunoassay device of claim 1, wherein the target material is at least one selected from the group consisting of an antibody, an antigen, a nucleic acid aptamer, a hapten, an antigen protein, a DNA, a DNA-binding protein, a hormone, a tumor-specific marker, and a tissue-specific marker.

8. The lateral flow immunoassay device of claim 1, wherein the sample is at least one selected from the group consisting of whole blood, plasma, serum, sputum, tears, mucus, nasal washes, nasal aspirate, breath, urine, semen, saliva, peritoneal washings, ascites, cystic fluid, meningeal fluid, amniotic fluid, leukocytes, peripheral blood mononuclear cells, buffy coat, glandular fluid, pancreatic fluid, lymph fluid, pleural fluid, nipple aspirate, bronchial aspirate, synovial fluid, joint aspirate, organ secretions, cells, cell extract, and cerebrospinal fluid.

9. A method for detecting a target material comprising:
introducing a sample containing a target material into a sample pad in the lateral flow immunoassay device according to claim 1.
